(19) United States
(12) Patent Application Publication
(10) Pub. No.: US 2017/0100481 A1
(43) Pub. Date: Apr. 13, 2017

(54) TRANSDERMAL PHARMACEUTICAL COMPOSITIONS WITH REDUCED SKIN IRRITATION

(71) Applicant: Noven Pharmaceuticals, Inc., Miami, FL (US)

(72) Inventors: Jeffrey KLEIN, Miami, FL (US); Puchun LIU, Miami, FL (US); Steven DINH, Miami, FL (US)

(73) Assignee: Noven Pharmaceuticals, Inc., Miami, FL (US)

(21) Appl. No.: 15/287,976

(22) Filed: Oct. 7, 2016

Related U.S. Application Data
(60) Provisional application No. 62/239,467, filed on Oct. 9, 2015.

(51) Int. Cl. A61K 47/28; A61K 9/70; A61K 47/22; A61K 31/4525

(52) U.S. Cl. CPC A61K 47/28; A61K 31/4525; A61K 9/7023; A61K 47/22

(57) ABSTRACT

Described are transdermal pharmaceutical compositions exhibiting reduced skin irritation potential comprising a steroid and an antioxidant, useful for reducing the skin irritation associated with the use of transdermal drug delivery compositions. Methods of making and using the compositions also are described.

### CROSS-REFERENCE TO RELATED APPLICATIONS

This application claims priority under 35 USC §119 to U.S. Provisional Application 62/239,467 filed Oct. 9, 2015, which is incorporated herein by reference in its entirety.

### FIELD

This invention relates generally to pharmaceutical compositions for transdermal drug delivery, and more particularly to transdermal pharmaceutical compositions that exhibit reduced skin irritation. Methods of making and using the compositions also are described.

### BACKGROUND

The use of transdermal pharmaceutical compositions to administer an active agent through the skin or mucosa of a subject is well known. Non-occlusive transdermal compositions typically incorporate the active agent into a carrier composition with volatile components that evaporate after application, such as liquid, gel, and emulsion compositions, while occlusive transdermal compositions typically incorporate the active agent into a carrier composition with at least some non-volatile components that do not evaporate after application, such as film-forming compositions or polymeric and/or pressure-sensitive adhesive compositions.

Many factors influence the design and performance of transdermal pharmaceutical compositions, such as the individual drugs themselves, the physical/chemical characteristics of the composition's components and the performance/behavior relative to other components once combined, external/environmental conditions during manufacturing and storage thereafter, the properties of the topical site of application, the desired rate of drug delivery and onset, the drug delivery profile, and the intended duration of delivery.

Transdermal pharmaceutical compositions may be associated with skin irritation. The nature and extent of skin irritation may vary with the drug and other components formulated in the compositions. For occlusive compositions, the duration of use of the system (e.g., how long a given system is left in place) also may impact the nature and extent of skin irritation. Even when a transdermal pharmaceutical composition exhibits a good therapeutic effect, skin irritation may undermine patient compliance and/or discourage the prescription or use of a given transdermal pharmaceutical composition.

While agents for reducing skin irritation associated with the use of transdermal pharmaceutical compositions are known, such agents may impact the performance of transdermal pharmaceutical compositions, such as by reacting with the drug or other components formulated in the composition, impacting the drug delivery profile, reducing drug delivery to an ineffective level, and/or impacting the physical properties of the composition (e.g., the adhesion and wear properties of an occlusive system). Thus, there remains a need for transdermal pharmaceutical compositions that exhibit reduced skin irritation.

### SUMMARY

Described are transdermal pharmaceutical compositions that exhibit reduced skin irritation, comprising a steroid and an antioxidant.

In some embodiments, a transdermal pharmaceutical composition as described herein comprises a drug to be delivered transdermally, and a steroid and an antioxidant in amounts effective to reduce the skin irritation potential of the composition. The steroid may be one or more selected from the group consisting of estradiol, estriol, β-sitosterol, stigmasterol, ergosterol, cholesterol, campersterol, and lanosterol. The antioxidant may be one or more selected from the group consisting of quercetin, α-bisobolol, rosamarinic acid, puerarin, hamamelitanin, aluminum acetate, curcumin, glycyrrhizin, retinol acetate, 1-ascorbyl palmitate, tocopherol, calcipotriol, tacalcitol, resveratrol, silibinin, ginsenoside, squalene, aluminum hydroxide, titanium oxide, ethylene diamine tetraacetic acid (EDTA), pantethine, and tranexamic acid. In specific embodiments, the steroid and antioxidant are selected from the group consisting of (i) estradiol and quercetin, (ii) estradiol and α-bisobolol, (iii) ergosterol and quercetin, (iv) stigmasterol and quercetin, (v) cholesterol and quercetin, (vi) cholesterol and resveratrol, (vii) cholesterol and silibinin, (viii) β-sitosterol and quercetin, (ix) β-sitosterol and silibinin, and (x) β-sitosterol and resveratrol. In other specific embodiments, the antioxidant comprises quercetin or α-bisobolol and/or the steroid comprises estradiol and the antioxidant comprises quercetin.

In some embodiments, the steroid and antioxidant exhibit a skin irritation reducing effect that is greater than that of the steroid or antioxidant alone. In some embodiments, neither the steroid nor antioxidant alone exhibit a skin irritation reducing effect. In some embodiments, neither the steroid nor antioxidant are present in an amount effective for systemic effect. In specific embodiments where the composition is a non-occlusive or film-forming composition, the steroid is present in an amount of from about 5 to about 200 mg/mL based on the total volume of the composition and/or the antioxidant is present in an amount of from about 0.05 to about 10 mg/mL based on the total volume of the composition.

In specific embodiments, the composition is a transdermal drug delivery system in the form of a flexible finite system for topical application. In some embodiments, the composition comprises amounts of the steroid and antioxidant effective to reduce skin irritation associated with use of the system over an application period of at least one day, at least three days, or at least seven days.

In specific embodiments, the steroid and antioxidant are present in a skin-contacting layer of the system. In specific embodiments, the steroid and antioxidant are present in a drug-containing polymer matrix layer of the system. In further specific embodiments, the steroid is present in an amount of from about 5 to about 200 mg/mL based on the total wet volume of the drug-containing polymer matrix and/or the antioxidant is present in an amount of from about 0.05 to about 10 mg/mL based on the total wet volume of the drug-containing polymer matrix.

Also provided are methods of reducing the skin irritating effects of a transdermal pharmaceutical composition, comprising formulating a transdermal pharmaceutical composition with a steroid and an antioxidant in amounts effective to reduce the skin irritation potential of the composition.

Also provided are methods of reducing the skin irritating effects of transdermal drug delivery, comprising administering to a subject in need of transdermal drug delivery a transdermal pharmaceutical composition comprising a steroid and an antioxidant in amounts effective to reduce the skin irritation potential of the composition. Also provided are transdermal pharmaceutical compositions as described herein for use in methods of reducing the skin irritating effects of transdermal drug delivery, or for use in methods of transdermally delivering a drug with reduced skin irritation effects.

Also provided are methods of making a transdermal pharmaceutical composition with reduced skin irritation potential, comprising preparing a transdermal pharmaceutical composition with a drug to be delivered transdermally, and a steroid and an antioxidant in amounts effective to reduce the skin irritation potential of the composition.

### BRIEF DESCRIPTION OF THE DRAWINGS

FIG. 1 A-C show the drug flux through human skin of paroxetine from different formulations described in the examples. Each of FIGS. 1A-C represents the drug flux through a given skin sample.

### DETAILED DESCRIPTION

Described are transdermal pharmaceutical compositions that exhibit reduced skin irritation associated with the use of transdermal pharmaceutical compositions.

### DEFINITIONS

Technical and scientific terms used herein have the meanings commonly understood by one of ordinary skill in the art to which the present invention pertains, unless otherwise defined. Reference is made herein to various methodologies known to those of ordinary skill in the art. Publications and other materials setting forth such known methodologies to which reference is made are incorporated herein by reference in their entireties as though set forth in full. Any suitable materials and/or methods known to those of ordinary skill in the art can be utilized in carrying out the present invention. However, specific materials and methods are described. Materials, reagents and the like to which reference is made in the following description and examples are obtainable from commercial sources, unless otherwise noted.

As used herein, the singular forms “a,” “an,” and “the” designate both the singular and the plural, unless expressly stated to designate the singular only.

The term “about” means that the number comprehended is not limited to the exact number set forth, and is intended to refer to values substantially around the quoted value while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

The phrase “substantially free” as used herein means that the described composition (e.g., polymer matrix, etc.) comprises less than about 5%, less than about 3%, or less than about 1% by weight, based on the total weight of the composition at issue, of the excluded component(s).

As used herein, “subject” denotes any mammal, including humans. For example, a subject may be suffering from or at risk of developing a condition that can be diagnosed, treated or prevented with a drug as described herein, or may be taking a drug for other purposes.

As used herein “drug” denotes any pharmaceutically or physiologically active agent, whether or not approved for use to prevent or treat a specific disease, disorder, or condition, including any agent having diagnostic, prophylactic, therapeutic, pharmacological, physiological, or cosmetic effect, including diagnostic agents, therapeutic agents, insect repellents, pesticides, sun screens, cosmetic agents, etc.

As used herein, “transdermal pharmaceutical composition” refers to a pharmaceutical composition formulated to deliver drug through the skin or mucosa of a subject, and includes non-occlusive and occlusive transdermal pharmaceutical compositions. As used herein, a “non-occlusive transdermal pharmaceutical composition” refers to a composition that does not substantially occlude the surface of the skin or mucosa to which it is applied. In a typical non-occlusive transdermal composition, the drug is incorporated into a carrier composition with volatile components that evaporate after application, such as liquid, gel, ointment, and emulsion compositions. As used herein, an “occlusive transdermal pharmaceutical compositions” refers to a composition that substantially occludes the surface of the skin or mucosa to which it is applied, such as a film-forming composition or transdermal patch-type system (also referred to herein as a “transdermal drug delivery system”).

As used herein, “transdermal drug delivery system” refers to a composition in a “flexible, finite form,” which refers to a substantially solid form capable of conforming to a surface with which it comes into contact, and capable of maintaining contact so as to facilitate topical application. Such systems in general are known in the art and commercially available, such as transdermal drug delivery patches.

As used herein, the terms “topical” and “topically” mean application to a skin or mucosal surface of a mammal, while the terms “transdermal” and “transdermal” connote passage through the skin or mucosa (including oral, buccal, nasal, rectal and vaginal mucosa), into systemic circulation. Thus, the systems described herein may be applied topically to a subject to achieve transdermal delivery of a drug.

As used herein, the phrases “therapeutically effective amount” and “therapeutic level” mean that drug dosage or plasma concentration in a subject, respectively, that provides the specific effect for which the drug is administered in a subject receiving the treatment. It is emphasized that a therapeutically effective amount or therapeutic level of a drug will not always be effective in achieving the intended effect, even though such dosage is deemed to be a therapeutically effective amount by those of skill in the art. For convenience only, exemplary dosages, drug delivery amounts, therapeutically effective amounts and therapeutic levels are provided below with reference to adult human subjects. Those skilled in the art can adjust such amounts in accordance with standard practices as needed to treat a specific subject and/or condition/disease.

As used herein “flux” is defined as the percutaneous absorption of drugs through the skin, and is described by Fick's first law of diffusion:

J=−D(dCₘ/dx),

where J is the flux in g/cm²/sec, D is the diffusion coefficient of the drug through the skin in cm²/sec and dCₘ/dx is the concentration gradient of the active agent across the skin or mucosa.

### Reduced Irritation Compositions

Described herein are transdermal pharmaceutical compositions comprising agents that reduce skin irritation associated with the use of transdermal pharmaceutical compositions. The agents may include a steroid and an anti-oxidant. It has been surprisingly discovered that such agents can reduce the skin irritation potential of a transdermal pharmaceutical composition while still achieving sufficient flux of drug through the skin to achieve the intended effect, e.g., without unduly inhibiting the efficacy of the compositions. In specific embodiments, while neither the steroid or anti-oxidant alone would be effective to reduce the skin irritation potential of the composition, the combination of the steroid and anti-oxidant is effective to reduce the skin irritation potential of the composition. In other specific embodiments, while neither the steroid or anti-oxidant alone would be effective to reduce the skin irritation potential of the composition in the amounts used, the combination of the steroid and anti-oxidant in the amounts used is effective to reduce the skin irritation potential of the composition.

The skin irritation potential of a transdermal pharmaceutical composition can be assessed by methods known in the art, such as by animal testing, such as by using a rabbit skin irritation test and scoring in accordance with the Primary Irritation Index (PII), as set forth in ASTM F719-81 (“Standard Practice for Testing Biomaterials in Rabbits for Primary Skin Irritation”) and illustrated in the examples.

Examples of steroids suitable for use as a skin irritation reducing agent include estradiol, estriol, β-sitosterol, stigmasterol, ergosterol, cholesterol, campersterol, and lanosterol.

In some embodiments, the amount of steroid used is not effective for systemic effect. That is, while the amount used is effective to reduce the skin irritation potential of the composition at the site of application of the composition, it does not exhibit a systemic physiological effect on the subject. In some embodiments, the steroid is used in an amount of from about 5 to about 200 mg/mL, including from about 10 to about 50 mg/mL, based on the total volume of the transdermal pharmaceutical composition (for non-occlusive or film-forming compositions) or based on the total wet volume of the polymer matrix (e.g., before drying to remove volatile processing solvents) (for transdermal drug delivery systems).

Examples of antioxidants suitable for use as a skin irritation reducing agent include quercetin, α-bisobolol, rosamarinic acid, puerarin, hamamelitanin, aluminum acetate, curcumin, glycyrrhizin, retinol acetate, 1-ascorbyl palmitate, tocopherol, calcipotriol, tacalcitol, resveratrol, silibinin, ginsenoside, squalene, aluminum hydroxide, titanium oxide, ethylene diamine tetraacetic acid (EDTA), pantethine, and tranexamic acid.

In some embodiments, the amount of antioxidant used is not effective for systemic effect. That is, while the amount used is effective to reduce the skin irritation potential of the composition at the site of application of the composition, it does not exhibit a systemic physiological effect on the subject. In some embodiments, the antioxidant is used in an amount of from about 0.05 to about 10 mg/mL, including from about 0.05 to about 1.0 mg/mL, and from about 0.1 to about 0.5 mg/mL, based on the total volume of the transdermal pharmaceutical composition (for non-occlusive or film-forming compositions) or based on the total wet volume of the polymer matrix (e.g., before drying to remove volatile processing solvents) (for transdermal drug delivery systems).

Any one or more steroid(s) can be used with any one or more antioxidant(s). If a combination of steroid(s) and/or antioxidant(s) is used, the total amount of steroid(s) and/or antioxidant(s) typically will be within the ranges-set forth above.

In specific embodiments, a composition exhibiting reduced skin irritation comprises a steroid and antioxidant selected from the group consisting of (i) estradiol and quercetin, (ii) estradiol and α-bisobolol, (iii) ergosterol and quercetin, (iv) stigmasterol and quercetin, (v) cholesterol and quercetin, (vi) cholesterol and resveratrol, (vii) cholesterol and silibinin, (viii) β-sitosterol and quercetin, (ix) β-sitosterol and silibinin, and (x) β-sitosterol and resveratrol.

### Transdermal Pharmaceutical Compositions

As noted above, transdermal pharmaceutical compositions that can be formulated as described herein with reduced skin irritation potential include non-occlusive transdermal pharmaceutical compositions and occlusive transdermal pharmaceutical compositions. Examples of non-occlusive transdermal pharmaceutical compositions include liquid, gel, ointment, cream, and emulsion compositions. Examples of occlusive transdermal pharmaceutical compositions include film-forming compositions that may be applied as a liquid, gel, ointment, cream, or emulsion and form a film on the skin or mucosal surface upon evaporation of volatile components, and patch-type systems which are discussed in more detail below. For any transdermal pharmaceutical composition, the drug may be formulated in any suitable form, including being dissolved, dispersed or suspended in a carrier composition. In embodiments where the drug is in solid form, the drug may be in any solid form, including any crystalline or amorphous form, and/or provided as a particles (e.g., drug-encapsulated particles, coated drug particle, drug nanoparticles, etc.).

Typically, the compositions are formulated to provide a diagnostically or therapeutically effective amount of drug, e.g., an amount of drug effective to exert the intended effect, based on the amount of composition to be applied. The amount of drug formulated in the composition may depend on the composition being prepared, the particular drug being formulated, the desired effect, and the duration for which the composition is to provide therapy. For most drugs, the passage of the drugs through the skin will be the rate-limiting step in delivery. Thus, the minimum amount of drug in the composition may be selected based on the amount of drug which passes through the skin from the composition and the in vivo drug level needed to exert the intended effect. The maximum amount of drug in a composition may be limited by the solubility of the drug in the composition or other chemical or physical properties and/or by the amount of drug to be delivered. The amount of drug in a composition can vary from as little as 0.001% or less to 95% or more.

The drug may be any drug suitable for local or transdermal delivery. For example, the drug may be or include any one or more of the drugs disclosed in U.S. Pat. No. 8,703,175 (granted Apr. 22, 2014), U.S. Pat. No. 8,865,207 (granted Oct. 21, 2014), U.S. Pat. No. 6,465,004 (granted Oct. 15, 2002), and U.S. Pat. No. 6,024,976 (granted Feb. 15, 2000), the entire contents of each of which are incorporated herein by reference as if they were expressly set forth herein.

In specific embodiments, the drug is associated with skin irritation. In other specific embodiments, the drug itself is not associated with skin irritation, but one or more other components of the composition is associated with skin irritation. While not wanting to be bound by theory, it has been postulated that drugs having a pKa value of greater than about 8 or less than about 4 may be associated with skin irritation. See Paudel et al., Ther. Deliv. 1(1): 109-31 (2010). Thus, in some embodiments, the drug is or includes a drug having a pKa value of greater than or equal to 8, or greater than 8. Additionally or alternatively, in some embodiments, the drug is or includes a drug having a pKa value of less than or equal to 4, or less than 4. Additionally or alternatively, in some embodiments, the drug is or includes a drug having a pKa value between 4 and 8.

In some embodiments, the drug is a non-steroidal drug. In specific embodiments, the drug is or includes paroxetine. In specific embodiments, the drug is or includes oxybutinin. In other specific embodiments, the drug is not oxybutynin, and the composition does not include oxybutinin. In some embodiments, the drug is not estradiol. In some embodiments, the drug is not estriol. In some embodiments, the drug is not β-sitosterol. In some embodiments, the drug is not stigmasterol. In some embodiments, the drug is not ergosterol. In some embodiments, the drug is not cholesterol. In some embodiments, the drug is not campersterol. In some embodiments, the drug is not lanosterol.

In some embodiments, the composition may include a skin penetration enhancer. The term “enhancer” as used herein refers to substances used to increase permeability and/or accelerate the delivery of an active agent through the skin or mucosa. Enhancers suitable for use in transdermal pharmaceutical compositions are known in the art, and may depend on the drug being formulated and the specific type of composition (e.g., liquid, gel, film-forming, patch, etc.). Typically, an enhancer (or combination thereof) may be used in a transdermal pharmaceutical compositions in an amount of up to about 50% by weight of the composition, or in any amount sufficient to yield a permeation-enhancing effect without rendering the composition as a whole unsuitable for its intended purpose.

In some embodiments, the composition may include one or more other pharmaceutically acceptable additives or excipients frequently used in such compositions. Such additives and excipients include diluents, stabilizers, buffering agents, biocides, humectants, anti-irritants, antioxidants, preservatives, flavoring agents, colorants, pigments and the like. Such substances can be present in any amount sufficient to impart the desired properties to the composition without rendering the composition as a whole unsuitable for its intended purpose. Typically, such additives or excipients are used in amounts up to 25% of the composition.

### Transdermal Drug Delivery Systems

In specific embodiments, the transdermal pharmaceutical composition is a transdermal drug delivery system. The transdermal drug delivery systems may be of any type known in the art, including drug-in-adhesive matrix-type or reservoir-type systems, and may comprise one or more drug-containing layers, reservoir layers, pressure-sensitive adhesive layers (whether or not containing drug), overlay layers, etc., as well as a backing and removable release liner. When present, the skin irritation-reducing agents as described herein may be present in any layer, but typically are present in a skin-contacting layer, such as a skin-contacting layer of a drug-in-adhesive matrix-type system or a skin-contacting layer of a reservoir-type system. Nevertheless, the agents may be formulated in any layer as long as the agents are able to contact the skin when the system is in use, such by migrating to a skin-contacting layer and coming into contact with the skin during use. In some embodiments one or more of the agents may be in different layers, such as a steroid being formulated in a skin-contacting layer and an antioxidant being formulated in a non-skin-contacting layer (or vice versa), as long as the agents are able to contact the skin when the system is in use, such as where the agent formulated in a non-skin-contacting layer is able to migrate to a skin-contacting layer and come into contact with the skin during use.

A drug-in-adhesive matrix-type system typically includes a drug-containing polymer matrix, which refers to a polymer composition which contains one or more drugs and one or more polymers, such as one or more pressure-sensitive adhesive polymers and/or bioadhesive polymers. As used herein, “active surface area” means the surface area of the drug-containing polymer matrix of the transdermal drug delivery system. A polymer is an “adhesive” or “bioadhesive” if it has the properties of adhesiveness per se. Other polymers can function as an adhesive or bioadhesive by the addition of tackifiers, plasticizers, crosslinking agents or other excipients. Thus, in some embodiments, the drug-containing polymer matrix optionally comprises tackifiers, plasticizers, crosslinking agents or other additives known in the art.

As used herein, the term “pressure-sensitive adhesive” refers to a viscoelastic material which adheres instantaneously to most substrates with the application of very slight pressure and remains permanently tacky. As noted above, a polymer is a pressure-sensitive adhesive polymer if it has the properties of a pressure-sensitive adhesive per se. Other polymers may function as a pressure-sensitive adhesive by admixture with tackifiers, plasticizers or other additives. The term pressure-sensitive adhesive also includes mixtures of different polymers.

In some embodiments, the polymer matrix is a pressure-sensitive adhesive at room temperature and exhibits desirable physical properties, such as good adherence to skin, ability to be peeled or otherwise removed without substantial trauma to the skin, retention of tack with aging, etc. In some embodiments, the polymer matrix has a glass transition temperature (T_{g}), measured using a differential scanning calorimeter, of between about −70° C. and 0° C.

In some embodiments, the systems are “monolithic” or “monolayer” systems, such that the drug-containing polymer matrix layer is the only polymeric layer present other than the backing layer and the release liner, if present. In such embodiments, the polymer matrix functions as both the drug carrier and the means of affixing the system to the skin or mucosa. In such embodiments, the skin irritation-reducing agents as described herein are present in the drug-containing polymer matrix layer, which also is the skin-contacting layer.

In other embodiments, the systems are multi-layer systems that include one or more drug-containing polymer matrix layers, or one or more non-drug containing polymer matrix layers in addition to one or more drug-containing polymer matrix layers. In such embodiments, the one or more additional layers may impart desired adhesion, wear, or pharmacokinetic properties, for example. In such embodiments, the skin irritation-reducing agents as described herein may be present in any layer as discussed above, but typically are present in the skin-contacting layer, e.g., the layer adjacent the release liner (if present) prior to its removal.

In other embodiments, the systems are reservoir systems that include a reservoir and an adhesive layer for adhering the system to the skin. In such embodiments, the skin irritation-reducing agents as described herein may be present in any portion of the system (as discussed above), but typically are present in the skin-contacting layer, e.g., the adhesive layer adjacent the release liner (if present) prior to its removal, or in the reservoir.

Suitable polymers, adhesive, and excipients for use in transdermal drug delivery systems are known in the art. For example, suitable adhesive include solvent-based, hot melt and grafted adhesives, which may be used alone or in combinations, mixtures or blends. Examples include acrylic-based polymer(s), silicone-based polymer(s), rubbers, gums, polyisobutylenes, polyvinylethers, polyurethanes, styrene block copolymers, styrene/butadiene polymers, polyether block amide copolymers, ethylene/vinyl acetate copolymers, and vinyl acetate based adhesives.

For most drugs, the passage of the drugs through the skin will be the rate-limiting step in delivery. Thus, the minimum amount of drug in the system is selected based on the amount of drug which passes through the skin from the composition during the duration which the device is to provide therapy. Normally, the amount of drug in a system can vary from as little as 0.1% or less to about 50% or more of the layer in which it is incorporated. In a reservoir-type system, the reservoir composition may be comprised of substantially only drug (e.g., if the drug is a liquid), or of a highly concentrated (e.g., saturated) composition of drug in a liquid, gel, semi-solid or solid matrix.

A system may include a skin penetration enhancer as discussed above in one or more of the layers. An enhancer may be formulated in any layer(s) of the system, but typically is formulated in a drug-containing layer and/or a skin-contacting layer. Typically, an enhancer (or combination thereof) may be used in a transdermal drug delivery system in an amount of up to about 30% by weight, based on the dry weight of the layer in which it is formulated, such as from about 0.1% to about 15%, or in any amount sufficient to yield a permeation-enhancing effect.

A system may include one or more other pharmaceutically acceptable additives or excipients frequently used in such systems, which may be present in any one or more layers of the system. Such additives include tackifying agents such as aliphatic hydrocarbons, mixed aliphatic and aromatic hydrocarbons, aromatic hydrocarbons, substituted aromatic hydrocarbons, hydrogenated esters, polyterpenes, silicone fluid, mineral oil and hydrogenated wood rosins. Additional additives include binders such as lecithin which “bind” the other ingredients, or rheological agents (thickeners) containing silicone such as fumed silica, reagent grade sand, precipitated silica, amorphous silica, colloidal silicon dioxide, fused silica, silica gel, quartz and particulate siliceous materials commercially available as Syloid®, Cabosil®, Aerosil®, and Whitelite®, for purposes of enhancing the uniform consistency or continuous phase of the composition or coating. Other additives and excipients include diluents, stabilizers, fillers, clays, buffering agents, biocides, humectants, anti-irritants, antioxidants, preservatives, plasticizing agents, crosslinking agents, flavoring agents, colorants, pigments and the like. Such substances can be present in any one or more layers, and can be present in any amount sufficient to impart the desired properties to the layer at issue or system as a whole. Typically, such additives or excipients are used in amounts up to 25% based on the dry weight of the layer(s) in which they are formulated, such as from about 0.1% to about 10%.

As noted above, transdermal drug delivery systems typically include a backing and a removable release liner to protect and/or anchor the system or its components during manufacturing as described herein, or thereafter, and to enable handling and transportation. Suitable materials for use as backings and release liners are known in the art.

An exemplary general method of preparing a transdermal drug delivery system is as follows:

Appropriate amounts of polymer(s), solvent(s), enhancer(s), and organic solvent(s) (for example toluene) are combined and thoroughly mixed together in a vessel. Drug is added to the mixture and agitation is carried out until the drug is uniformly mixed in.
The formulation is then transferred to a coating operation where it is coated onto a protective release liner at a controlled specified thickness. The coated product is then passed through an oven in order to drive off all volatile processing solvents.
The dried product on the release liner is then joined to the backing material and wound into rolls for storage.
Thereafter, desired size and shape delivery systems are prepared by die-cutting or the like, from the rolled laminate and then packaged.

### Therapeutic Methods

As noted above, the compositions described herein can be used to transdermally administer any drug, for any desired effect, including diagnostic, prophylactic, therapeutic, or cosmetic effect. In such methods, a composition as described herein is applied to the skin or mucosa of the subject. In some embodiments using a transdermal drug delivery system, a system is applied for about one day. In other embodiments using a transdermal drug delivery system, a system is applied for more than one day, such as for two days, three days, four days, five days, six days, seven days, or longer. In specific embodiments, the use of a composition as described herein, comprising skin irritation reducing agents as described herein, reduces the skin irritation that otherwise would be associated with the transdermal delivery of the drug from a comparable composition formulated without the skin irritation reducing agents, e.g., formulated without the steroid(s) and antioxidant(s). That is, the skin irritation reducing agents are effective to reduce the skin irritation potential of the composition. As noted above, the skin irritation potential of a composition can be assessed by methods known in the art, such as by animal testing, such as by using a rabbit skin irritation test and scoring in accordance with the Primary Irritation Index (PII), as set forth in ASTM F719-81 and illustrated in the examples.

Thus, also provided herein are methods of reducing the skin irritating effects of a transdermal pharmaceutical composition, comprising formulating a transdermal pharmaceutical composition with skin irritation reducing agents including a steroid and an antioxidant effective to reduce the skin irritation potential of the composition, and methods of reducing the skin irritating effects of transdermal drug delivery, comprising administering to a subject in need of transdermal drug delivery a transdermal pharmaceutical composition comprising a steroid and an antioxidant effective to reduce the skin irritation potential of the composition.

The following specific examples are included as illustrative of the compositions described herein. These example are in no way intended to limit the scope of the invention. Other aspects of the invention will be apparent to those skilled in the art to which the invention pertains.

### Examples

Paroxetine was used as a representative drug associated with skin irritation when delivered transdermally. Saturated compositions of paroxetine formulated in mineral oil (1:5) were prepared using multiple steroid and/or antioxidant combinations and assessed using a rabbit skin irritation model. Rabbits were scored using the primary irritation index (PII), based on erythema and edema scoring at 24 hours following skin exposure to the formulations. Because PII scores can vary across individual animals, the same rabbits (n=3) were used for multiple treatments to reduce variability.

Estradiol Alone

| Formulation | PII (24 hours) |
|---|---|
| Paroxetine in mineral oil (1:5) | 5.3 |
| Paroxetine in mineral oil (1:5) | 5.1 |
| with 0.4 mg/mL estradiol | |

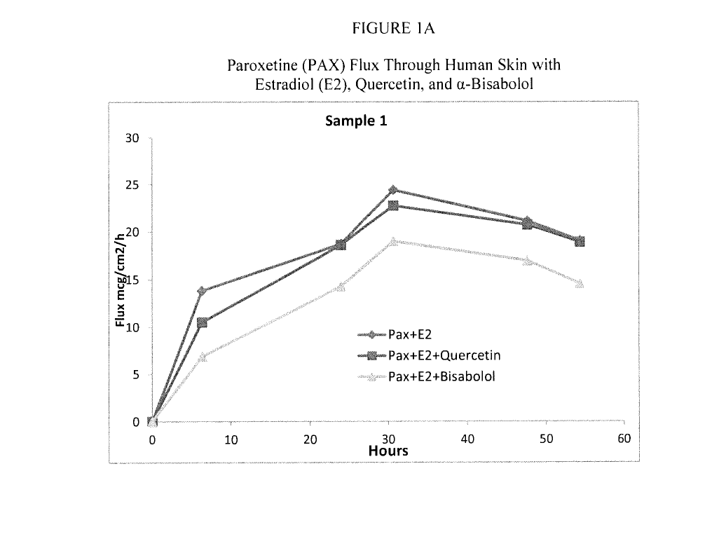

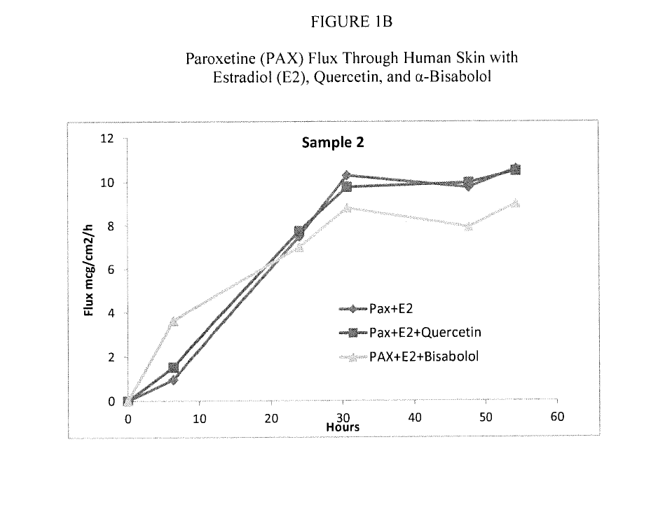

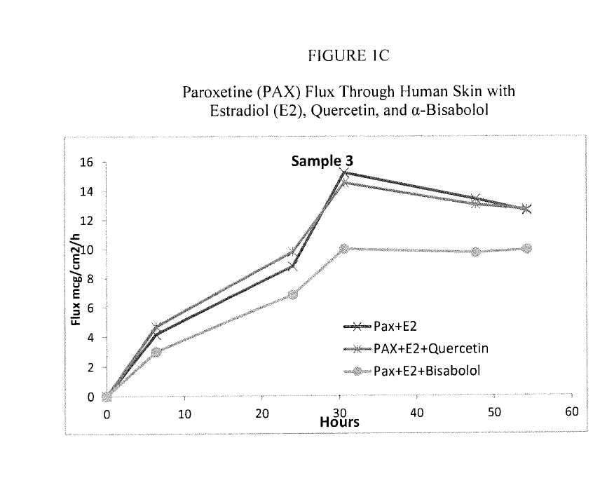

The use of 0.4 mg/mL estradiol was determined to not have an irritation-reducing effect. In vitro flux studies using human skin showed that paroxetine was delivered through the skin in the presence of 0.4 mg/mL estradiol (FIG. 1A-C).

Estradiol and Quercetin

| Formulation | PII (24 hours) |
|---|---|
| Paroxetine in mineral oil (1:5) | 1.7 |
| with 0.4 mg/mL estradiol | |
| Paroxetine in mineral oil (1:5) | 0.9 |
| with 0.4 mg/mL estradiol and | |
| 0.1 mg/mL quercetin | |
| Paroxetine in mineral oil (1:5) | 0.1 |
| with 0.4 mg/mL estradiol and | |
| 0.2 mg/mL quercetin | |

The use of quercetin with estradiol was determined to have a concentration-dependent irritation-reducing effect (based on the concentration of quercetin). In vitro flux studies using human skin showed that paroxetine was delivered through the skin in the presence of 0.4 mg/mL estradiol and 0.2 mg/mL quercetin (FIG. 1A-C). This experiment indicates that the use of a combination of skin irritation reducing agents as described herein could be useful for reducing the skin irritation of a transdermal drug delivery system that is applied to the skin for one day or longer.

A 7-day study with the estradiol and high concentration quercetin/estradiol formulations showed a PII value of 2.3 for the estradiol formulation versus 1.3 for the high concentration quercetin/estradiol formulation. This experiment indicates that the use of a combination of skin irritation reducing agents as described herein could be useful for reducing the skin irritation of a transdermal drug delivery system that is applied to the skin for longer than one day, such as for 7 days.

Estradiol and α-Bisobolol

| Formulation | PII (24 hours) |
|---|---|
| Paroxetine in mineral oil (1:5) | 2.4 |
| with 0.4 mg/mL estradiol | |
| Paroxetine in mineral oil (1:5) | 1.9 |
| with 0.4 mg/mL estradiol and | |
| 0.5% α-bisobolol | |
| Paroxetine in mineral oil (1:5) | 1.8 |
| with 0.4 mg/mL estradiol and | |
| 1.0% α-bisobolol | |

The use of α-bisobolol with estradiol was determined to have a concentration-dependent irritation-reducing effect (based on the concentration of α-bisobolol). In vitro flux studies using human skin showed that paroxetine was delivered through the skin in the presence of 0.4 mg/mL estradiol and 1% α-bisobolol (FIG. 1A-C), with no statistically significant affect on flux.

Ergosterol and Quercetin

| Formulation | PII (24 hours) |
|---|---|
| Paroxetine in mineral oil (1:5) | 2.8 |
| with 0.2 mg/mL quercetin | |
| Paroxetine in mineral oil (1:5) | 2.6 |
| with 2 mg/mL ergosterol | |
| Paroxetine in mineral oil (1:5) | 1.9 |
| 0.2 mg/mL quercetin and | |
| 2 mg/mL ergosterol | |

The use of quercetin or ergosterol alone was determined not to have an irritation-reducing effect; however, their use in combination was determined to have an irritation-reducing effect.

Stimasterol and Quercetin

| Formulation | PII (24 hours) |
|---|---|
| Paroxetine in mineral oil (1:5) | 2.4 |
| with 0.2 mg/mL quercetin | |
| Paroxetine in mineral oil (1:5) | 2.9 |
| with 2 mg/mL stigmasterol | |
| Paroxetine in mineral oil (1:5) | 2.1 |
| 0.2 mg/mL quercetin and | |
| 2 mg/mL stigmasterol | |

The use of quercetin or stigmasterol alone was determined not to have an irritation-reducing effect; however, their use in combination was determined to have an irritation-reducing effect.

What is claimed is:

1. A transdermal pharmaceutical composition comprising a drug to be delivered transdermally, and a steroid and an antioxidant in amounts effective to reduce the skin irritation potential of the composition.

2. The composition of claim 1, wherein the steroid comprises one or more selected from the group consisting of estradiol, estriol, β-sitosterol, stigmasterol, ergosterol, cholesterol, campersterol, and lanosterol.

3. The composition of claim 1, wherein the antioxidant comprises one or more selected from the group consisting of quercetin, α-bisobolol, rosamarinic acid, puerarin, hamamelitanin, aluminum acetate, curcumin, glycyrrhizin, retinol acetate, 1-ascorbyl palmitate, tocopherol, calcipotriol, tacalcitol, resveratrol, silibinin, ginsenoside, squalene, aluminum hydroxide, titanium oxide, ethylene diamine tetraacetic acid (EDTA), pantethine, and tranexamic acid.

4. The composition of claim 1, wherein the steroid and antioxidant are selected from the group consisting of (i) estradiol and quercetin, (ii) estradiol and α-bisobolol, (iii) ergosterol and quercetin, (iv) stigmasterol and quercetin, (v) cholesterol and quercetin, (vi) cholesterol and resveratrol, (vii) cholesterol and silibinin, (viii) β-sitosterol and quercetin, (ix) β-sitosterol and silibinin, and (x) β-sitosterol and resveratrol.

5. The composition of claim 1, wherein the antioxidant comprises quercetin or α-bisobolol.

6. The composition of claim 1, wherein the steroid comprises estradiol and the antioxidant comprises quercetin.

7. The composition of claim 1, wherein the steroid and antioxidant exhibit a skin irritation reducing effect that is greater than that of the steroid or antioxidant alone.

8. The composition of claim 1, wherein neither the steroid nor antioxidant alone exhibit a skin irritation reducing effect.

9. The composition of claim 1, wherein neither the steroid nor antioxidant are present in an amount effective for systemic effect.

10. The composition of claim 1, wherein the composition is a non-occlusive or film-forming composition and the steroid is present in an amount of from about 5 to about 200 mg/mL based on the total volume of the composition.

11. The composition of claim 1, wherein the composition is a non-occlusive or film-forming composition and the antioxidant is present in an amount of from about 0.05 to about 10 mg/mL based on the total volume of the composition.

12. A flexible, finite transdermal drug delivery system for topical application comprising the composition of claim 1.

13. The transdermal drug delivery system of claim 12, wherein the composition comprises amounts of the steroid and antioxidant effective to reduce skin irritation associated with use of the system over an application period of at least one day.

14. The transdermal drug delivery system of claim 12, wherein the composition comprises amounts of the steroid and antioxidant effective to reduce skin irritation associated with use of the system over an application period of at least three days.

15. The transdermal drug delivery system of claim 12, wherein the composition comprises amounts of the steroid and antioxidant effective to reduce skin irritation associated with use of the system over an application period of at least seven days.

16. The transdermal drug delivery system of claim 12, wherein the steroid and antioxidant are present in a skin-contacting layer of the system.

17. The transdermal drug delivery system of claim 12, wherein the steroid and antioxidant are present in a drug-containing polymer matrix layer of the system.

18. The transdermal drug delivery system of claim 17, wherein the steroid is present in an amount of from about 5 to about 200 mg/mL based on the total wet volume of the drug-containing polymer matrix.

19. The transdermal drug delivery system of claim 17, wherein the antioxidant is present in an amount of from about 0.05 to about 10 mg/mL based on the total wet volume of the drug-containing polymer matrix.

20. A method of reducing the skin irritating effects of a transdermal pharmaceutical composition, comprising formulating a transdermal pharmaceutical composition with a steroid and an antioxidant in amounts effective to reduce the skin irritation potential of the composition.

21. A method of reducing the skin irritating effects of transdermal drug delivery, comprising administering to a subject in need of transdermal drug delivery a transdermal pharmaceutical composition comprising a steroid and an antioxidant in amounts effective to reduce the skin irritation potential of the composition.

22. A method of making a transdermal pharmaceutical composition with reduced skin irritation potential, comprising preparing a transdermal pharmaceutical composition with a drug to be delivered transdermally, and a steroid and an antioxidant in amounts effective to reduce the skin irritation potential of the composition.
